(19) United States
(12) Patent Application Publication
(10) Pub. No.: US 2026/0069586 A1
(43) Pub. Date: Mar. 12, 2026

(54) METHODS FOR REDUCING WEIGHT AND PRESERVING OR INCREASING TISSUE LEAN MASS IN PATIENTS SUFFERING FROM CUSHING'S SYNDROME

(71) Applicant: Corcept Therapeutics Incorporated, Redwood City, CA (US)

(72) Inventor: Andreas Moraitis, Sunny Isles Beach, FL (US)

(73) Assignee: Corcept Therapeutics Incorporated, Redwood City, CA (US)

(21) Appl. No.: 19/323,835

(22) Filed: Sep. 9, 2025

Related U.S. Application Data
(60) Provisional application No. 63/693,007, filed on Sep. 10, 2024.

(51) Int. Cl. A61K 31/4745; A61P 3/04; A61P 3/10

(52) U.S. Cl. CPC A61K 31/4745; A61P 3/04; A61P 3/10

(57) ABSTRACT

Applicant discloses methods and uses of the heteroaryl ketone fused azadecalin compound relacorilant for treating patients suffering from endogenous hypercortisolism (CS) (including Cushing's syndrome and Cushing's Disease) effective reduce or prevent the loss of tissue lean mass; in embodiments, the methods and uses are effective to increase tissue lean mass as compared to the patient's tissue lean mass prior to administration of relacorilant. The methods and uses of relacorilant for treating patients suffering from CS are further effective to reduce body weight and waist circumference of the patient.

Therapeutic amounts of relacorilant may be between about 50 milligrams per day (mg/day) and up to about 800 mg/day, e.g., 100 mg/day, 200 mg/day, 300 mg/day, 400 mg/day, or 500 mg/day. The treatments may be oral treatments, and relacorilant may be administered with food, or without food. Relacorilant is (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridine-2-yl)methanone, which has the structure:

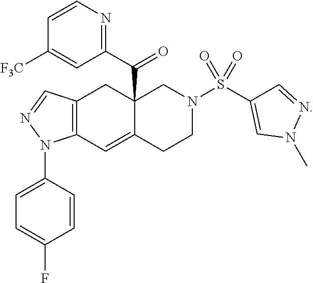 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

### CROSS-REFERENCE TO RELATED APPLICATION

This application claims priority to, and the benefit of, U.S. Provisional Patent Application No. 63/693,007, filed Sep. 10, 2024, which application is hereby incorporated by reference herein in its entirety.

### BACKGROUND

Cortisol is a steroid hormone secreted by the adrenal glands due to the action of adrenocorticotropin hormone (ACTH). ACTH is secreted by the pituitary gland under the control of the hypothalamic hormone corticotropin releasing factor (CRF). Cortisol action requires that cortisol bind to the glucocorticoid receptor (GR). Cushing's syndrome and Cushing's Disease are disorders of cortisol excess (hypercortisolism, termed herein “CS”). Long-term or excessive administration of glucocorticoids such as dexamethasone, prednisone, and others (which mimic cortisol action) can also cause hypercortisolism; such hypercortisolism is termed exogenous hypercortisolism or exogenous CS.

However, endogenous factors may also cause hypercortisolism. Endogenous hypercortisolism is a multisystem endocrine disorder characterized by prolonged exposure to excess cortisol activity. Such cortisol excess may be due to any of several possible causes, including excess secretion of adrenocorticotrophic hormone (ACTH) from the pituitary gland, excess cortisol secretion from adrenal glands, or secretion of cortisol or ACTH from non-cancerous tumors. Endogenous hypercortisolism, e.g. CS, is typically caused by a pituitary tumor, although endogenous hypercortisolism can also be caused by adrenal gland and other tumors. Where the excess cortisol is produced by the patient, the disease is termed endogenous hypercortisolism. Patients with endogenous hypercortisolism typically suffer metabolic complications such as hyperglycemia (often in the form of type 2 diabetes) and hypertension. Cushing syndrome, a severe form of endogenous hypercortisolism, and Cushing Disease (Cushing syndrome due to a pituitary tumor).

In addition to hypercortisolism, other symptoms and co-morbidities of endogenous hypercortisolism include hyperglycemia (and often diabetes), hypertension, disorders of the heart rhythm, weight gain (often including obesity, and may include characteristic fat deposition on the face, neck or back), skin stria, depression, hirsuteness, depression, muscle weakness, and loss of tissue lean mass (also termed “muscle lean mass”).

The first line of treatment for tumor-caused CS is surgical resection of the tumor. Still, patients who have persistent hypercortisolism post-surgery, who need CS management while awaiting surgery, or who have surgical contraindications, require a non-surgical therapeutic option to treat their cortisol excess and its effects.

A compound that modulates or affects cortisol binding to the GR is termed a GR modulator (GRM). Medical treatments for CS include GRMs such as, e.g., mifepristone; drugs that affect the production of cortisol (e.g., ketoconazole, levoketoconazole, and osilodrostat); and drugs that affect ACTH levels (e.g., pasireotide).

However, none of the available treatment methods specifically address the muscle weakness and loss of tissue lean mass caused by CS. In fact, even after successful treatment of CS, muscle weakness and loss of tissue lean mass may persist (see Vogel, Frederick et al J. Clin. Endocrinol Metab Vol. 105(12): e4490-e4498, 2020).

Excess cortisol results in muscle weakness and muscle tissue loss due to the catabolic effect glucocorticoids have on muscle cells. Cortisol binding to GRs in the muscle cell alters the metabolism of muscle lipids, carbohydrates, and proteins. This alteration then leads to the breakdown of muscle proteins, intramuscular fat accumulation, and atrophy of type 2 muscle fibers (see Jeddi, M, Shams, M. J Med Case Reports 16, 483, 2022). As a consequence, muscle weakness and muscle tissue loss is a prevalent symptom of CS, affecting up to 60% of patients (see Vogel, Frederick et al. J. Clin. Endocrinol Metab Vol. 105(12): e4490-e4498, 2020).

There is an unmet need for safe CS medications and treatment methods for treating muscle weakness and loss of tissue lean mass. Accordingly, in order to improve treatments for CS patients, improved methods and compositions that increase muscle mass and reduce or counteract muscle weakness are needed.

### SUMMARY

Patients suffering from Cushing's syndrome and Cushing's Disease (collectively “CS”)) typically suffer muscular weakness, including typically suffering loss of tissue lean mass. Weight gain (including, e.g., increased waist circumference) is also a serious comorbidity suffered by CS patients.

Disclosed herein are novel methods for treating CS which are effective to reduce loss of tissue lean mass over time as compared to average observed loss of tissue lean mass over time in patients suffering from CS, to reduce weight in patients suffering from CS by preserving or increasing tissue lean mass, and to improve muscle strength in patients suffering from CS. In embodiments, the novel methods for treating CS are effective to increase tissue lean mass as compared to baseline levels of tissue lean mass in patients suffering from CS. Disclosed herein are methods for reducing muscle weakness in a patient suffering from CS, as indicated by reduced time for the patient to stand up from a sitting position, as compared to the baseline time required to stand up from a sitting position. Disclosed herein are novel methods for treating CS which are effective to reduce body weight as compared to baseline levels of body weight in patients suffering from CS. Disclosed herein are novel methods for treating CS which are effective to reduce waist circumference as compared to baseline levels of waist circumference in patients suffering from CS. Disclosed herein are novel methods for treating CS effective to reduce weight in patients suffering from CS while preserving or increasing tissue lean mass in those patients. Disclosed herein are novel methods for treating CS effective to improve muscle strength in patients suffering from CS. Disclosed herein are novel methods for treating CS effective to preserve or to increase tissue lean mass in patients suffering from CS while those patients are receiving a weight-loss medication (e.g., a glucagon-like peptide (GLP-1) agonist).

The methods comprise administering to the subject an effective amount of the heteroaryl ketone fused azadecalin compound relacorilant, ((R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridine-2-yl)methanone), which has the structure:

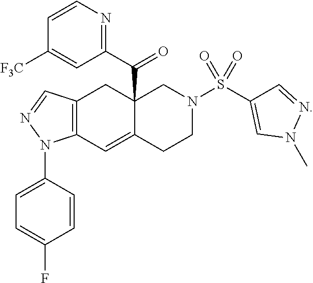 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

Heteroaryl ketone fused azadecalin compounds are disclosed in U.S. Pat. No. 8,859,774, the entire contents of which patent is hereby incorporated by reference in its entirety.

Applicant discloses herein uses of relacorilant for treating a patient suffering from CS effective to reduce the loss of tissue lean mass over time of the patient as compared to average observed loss of tissue lean mass over time in patients suffering from CS. In embodiments, the uses of relacorilant for treating a patient suffering from CS are effective to increase tissue lean mass as compared to baseline levels of tissue lean mass. Disclosed herein are uses of relacorilant for reducing muscle weakness in a patient suffering from CS, as indicated by reduced time for the patient to stand up from a sitting position, as compared to the baseline time required to stand up from a sitting position. Disclosed herein are novel uses of relacorilant for treating CS while reducing body weight as compared to baseline levels of body weight in patients suffering from CS. Disclosed herein are novel uses of relacorilant for treating CS while reducing waist circumference as compared to baseline levels of waist circumference in patients suffering from CS; and other uses of relacorilant in the treatment of patients suffering from CS.

In embodiments of the methods for treating CS while increasing tissue lean mass, administration of relacorilant for 22 weeks is effective to increase tissue lean mass in a patient suffering from CS by about 1.8% as compared to baseline tissue lean mass. In embodiments of the methods for treating CS while reducing body weight, administration of relacorilant for 22 weeks is effective to reduce body weight in a patient suffering from CS by at least about 2%, or at least about 3%, or at least about 5%, or at least about 10%, or at least about 20%, as compared to baseline body weight. In some cases, patient body weight was reduced by up to about 40% to 50% of their initial body weight. In embodiments of the methods for treating CS while reducing waist circumference as compared to baseline levels of waist circumference in patients suffering from CS, the administration of relacorilant for 22 weeks is effective to reduce waist circumference by about 1%, and up to about 2.9%. In embodiments of the methods for treating CS while reducing muscle weakness, administration of relacorilant for 22 weeks is effective to reduce the time required for a patient suffering from CS to stand up from a sitting position by about 1.5% as compared to the baseline time required for the patient to stand up from a sitting position.

In embodiments of the uses of relacorilant for treating CS while increasing tissue lean mass, use of relacorilant is effective to increase tissue lean mass in a patient suffering from CS patient by about 1.8% as compared to baseline tissue lean mass. In embodiments of the uses of relacorilant for treating CS while reducing body weight, use of relacorilant is effective to reduce body weight in a patient suffering from CS patient by at least about 2%, and up to about 3.4% as compared to baseline body weight. In embodiments of the uses of relacorilant for treating CS while reducing muscle weakness, use of relacorilant is effective to reduce the time required for a patient suffering from CS to stand up from a sitting position by about 1.5% as compared to the baseline time required for the patient to stand up from a sitting position.

In embodiments of the uses of relacorilant for treating CS while preserving tissue lean mass, or reducing tissue lean mass, relacorilant may be administered to a patient suffering from CS along with a weight-loss medication (e.g., a GLP-1 agonist (e.g., Dulaglutide (Trulicity®), Exenatide (Byetta®, Bydureon®), Liraglutide (Victoza®, Saxenda®), Lixisenatide (Adlyxin®), Semaglutide (Wegovy®, Ozempic®, Rybelsus®), Bupropion-naltrexone (Contrave®), Orlistat (Xenical®, Alli®), Phentermine-topiramate (Qsymia©), Setmelanotide (Imcivree©), or other weight-loss medication).

In embodiments, relacorilant is orally administered. In embodiments, relacorilant may be administered at daily doses of 50 milligrams (mg), 100 mg, 200 mg, 300 mg, 400 mg, or 500 mg. In embodiments, relacorilant may be administered with food, or with water, or with both food and water. In other embodiments, relacorilant may be administered without food. In embodiments, relacorilant may be administered to a fasted patient without food.

Accordingly, the present methods provide improved methods of treating CS patients suffering from loss of tissue lean mass (e.g., muscle mass), or for weight gain, or both. The present methods, which are shown herein to be effective to increase tissue lean mass in patients suffering from CS, are believed to treat muscle weakness in patients suffering from CS, are believed to increase the amounts of muscle tissue in such patients, and are believed to increase muscular strength in in patients suffering from CS. Thus, the present methods provide improved methods of treating patients suffering from CS and comorbidities associated with CS.

### BRIEF DESCRIPTION OF THE DRAWINGS

FIG. 1 shows results of relacorilant administration in patients suffering from CS, including demonstrating increased tissue lean mass percentage, decreased weight, decreased waist circumference, decreased tissue fat percentage, and reduced time to stand from a sitting position, all as compared to baseline. BL, baseline; DXA, Dual Energy X-Ray Absorptiometry; OL22 week 22 of the Open Label phase. Error bars: Standard deviation. Wilcoxon rank sum test P-values for the mean change from baseline shown.

FIG. 2 shows results of 22 weeks of relacorilant administration during the Open Label phase of the study demonstrating decreased body weight as compared to baseline, and decreased waist circumference as compared to baseline. LSM, least-mean square; SE, standard error; MMRM, mixed model for repeated measures. LSM and SE calculated using a linear MMRM. Error bars: SE of the mean. LSM and SE calculated using a linear MMRM. Wilcoxon rank sum test P-values for the mean change from BL shown. ns, not significant (P≥0.05); *, P<0.05; **, P<0.01; ***, P<0.001; ****, P<0.0001.

FIG. 3 shows that the improvements in body weight composition were maintained in patients who continued to receive relacorilant administration during the 12 week Randomized Withdrawal phase following the 22 week Open Label phase, but were not maintained in patients who received placebo for the 12 weeks following the 22 weeks of relacorilant administration during the Open Label phase. Body weight changes are as compared to baseline weight measured prior to beginning relacorilant administration. BL, baseline; LSM, least squares mean; SE, standard error. Error bars: SE of the mean. Wilcoxon signed-rank test P-values within each treatment arm.

FIG. 4A shows tissue fat mass percent as measured at baseline (BL) and week 22 of the Open Label phase (OL22). Tissue fat mass percent was significantly reduced at week 22 of the Open Label phase as compared to baseline (P<0.0001). The reduction in tissue fat mass percent was maintained in patients who continued to receive relacorilant administration during the 12 week Randomized Withdrawal phase (RW12) following the 22 week Open Label phase (tissue fat mass percent measured at the end of the 12 week RW phase), but was not maintained in patients who received placebo in the RW phase. (In this figure and in subsequent figures, BL indicates baseline; DXA indicates dual X-ray absorptiometry; OL indicates Open Label phase of the study; RW indicates Randomized Withdrawal phase of the study; Error bars indicate standard deviation; tissue fat mass and tissue lean mass were measured by DXA; Wilcoxon rank sum test P-values for the mean change from baseline are shown.)

FIG. 4B shows tissue lean mass percent as measured at baseline (BL) and week 22 of the OL phase (OL22). Tissue lean mass percent was significantly increased at week 22 of the OL phase as compared to baseline (P<0.0001). The increase in tissue lean mass percent was maintained in patients who continued to receive relacorilant administration during the 12 week Randomized Withdrawal phase (RW12) following the 22 week OL phase (tissue lean mass percent measured at the end of the 12 week RW phase), but was not maintained in patients who received placebo in the RW phase.

FIG. 4C shows patient waist circumference as measured at BL and week 22 of OL (OL22). Patient waist circumference was significantly reduced at week 22 of the OL phase as compared to BL (P<0.0001). The reduction in waist circumference was maintained in patients who continued to receive relacorilant administration during the 12 week RW phase (RW12) following the 22 week OL phase (waist circumference measured at the end of the 12 week RW phase), but was not maintained in patients who received placebo in the RW phase.

FIG. 4D shows patient body weight as measured at BL and week 22 of the OL phase (OL22). Patient body weight was significantly reduced at week 22 of the OL phase as compared to BL (P<0.0001). The reduction in body weight was maintained in patients who continued to receive relacorilant administration during the 12 week RW phase (RW12) following the 22 week OL phase (measured at the end of the 12 week RW phase). The body weight reduction was also maintained in patients who received placebo during the 12 week RW phase.

### DETAILED DESCRIPTION

### Introduction

Patients suffering from endogenous hypercortisolism (CS) typically experience muscle weakness, and loss of tissue lean mass (muscle lean mass). Patients suffering from CS typically gain weight as compared to their weight before diagnosis. Patients suffering from CS typically suffer from muscle weakness, as compared to their bodily strength before diagnosis. Patients suffering from CS typically suffer from loss of muscle mass, indicated by loss of tissue lean mass, as compared to their tissue lean mass before diagnosis. Such changes in body composition are typical symptoms of the disorder. Treatments that ameliorate, or reduce, or prevent, or reverse loss of tissue lean mass in patients suffering from CS would provide great benefit to those patients. Treatments that ameliorate, or reduce, or prevent, or reverse weight gain, or reduce or reverse loss of tissue lean mass, or other changes in body composition, in patients suffering from CS would provide great benefit to those patients. Applicant discloses such treatments herein.

Patients suffering from CS typically experience weight gain. Treatments that reduce, or prevent, or reverse weight gain in patients suffering from CS would provide great benefit to those patients. Applicant discloses such treatments herein.

### Definitions

As used herein, the term “CS” refers to all forms of endogenous hypercortisolism, including Cushing's syndrome and Cushing's Disease.

As used herein, the term “patient” refers to a human that is or will be receiving, or has received, medical care for a disease or condition, such as, e.g., CS.

As used herein, the term “effective amount” or “therapeutic amount” refers to an amount of a pharmacological agent effective to treat, eliminate, or mitigate at least one symptom of the disease being treated. In some cases, “therapeutically effective amount” or “effective amount” can refer to an amount of a functional agent or of a pharmaceutical composition useful for exhibiting a detectable therapeutic. The effect can be detected by any assay method known in the art.

As used herein, the terms “significant” and “significantly”, as used to characterize a change in a value or an effect, indicate a change in a value or a change in an effect that would be expected to have a clinical effect. Significance may be indicated by a “P” value, where P<0.05 may be considered statistically significant.

As used herein, the terms “substantial” and “substantially”, as used to characterize a change in a value or an effect, indicate a change in a value or a change in an effect that would be expected to have a clinical effect.

As used herein, the term “about”, as used to refer to a value A, as in, for example, “about A”, indicates that the value may be within a range of 10% of A; that is, the value may be within the range of 0.9×A to 1.1×A, inclusive (where “×” indicates multiplication).

As used herein, the term “baseline” refers to an initial measurement, or condition, determined at a time prior to the beginning of a treatment such as administration of an active agent such as relacorilant.

As used herein, the terms “administer,” “administering,” “administered” or “administration” refer to providing a compound or a composition (e.g., one described herein), to a subject or patient. For example, a compound or composition may be administered orally to a patient (i.e., the subject receives the compound or composition via the mouth, as a pill, capsule, liquid, or in other form suitable for administration via the mouth. Oral administration may be buccal (where the compound or composition is held in the mouth, e.g., under the tongue, and absorbed there). Administration may be by other modes of administration, e.g., by injection, by inhalation, by absorption into the skin from an implant containing the compound or composition), or by other route.

As used herein, the term “tissue fat mass” refers to the amount of fat (lipid-triglycerides, phospholipid membranes, etc.) as measured in a subject by DXA measurement. Changes in body composition typically found in Cushing's syndrome patients include changes (increases) in tissue fat mass.

As used herein, the term “tissue lean mass” refers to the amount of lipid-free body tissue, other than fat and other than bone, as measured in a subject by DXA measurement. This term also refers to muscle lean mass. Changes in body composition typically found in Cushing's syndrome patients include changes (decreases) in tissue lean mass.

As used herein, the term “loss in tissue lean mass over time” refers to the reduction in tissue lean mass in a patient suffering from CS between an initial time period and a later time period. Such a time period may be, for example, one month, or two months, or three months, or six months, or nine months, or one year, or may be other suitable time period. An average of the loss of tissue lean mass over time may be determined from the loss in tissue lean mass over time for a group of CS patients (e.g., at least five or at least ten or more CS patients). The loss of tissue lean mass over time may be determined for any one CS patient by measuring tissue lean mass at an initial time (e.g., prior to beginning treatment for CS) and then measuring that patient's tissue lean mass at a later time and determined the difference between the two measurements. Such a later time after that initial time may be, for example, one month later, or two months later, or three months later, or six months later, or nine months later, or one year later, or may be other suitable time period after the initial time.

As used herein, the term “DXA” refers to dual energy X-ray absorptiometry. The use of DXA in body composition measurements are discussed, for example, in Messina et al., “Body composition with dual energy X-ray absorptiometry: from basics to new tools”, Quantitative Imaging in Medicine and Surgery, Vol. 10:8 (Aug. 1, 2020).

As used herein, the term “with food” refers to administration of a drug within about one half hour (before or after) of the consumption of a meal by the patient.

As used herein, the term “without food” refers to administration of a drug to a patient who has not consumed a meal within about one half hour before such drug administration, and who does not consume a meal within about one half hour after the drug administration to the patient.

As used herein, the terms “fasted”, “fasted patient”, and the like, refer to a patient who has not consumed a meal for at least one hour prior to administration of a drug.

As used herein, the term “combination therapy” refers to the administration of at least two pharmaceutical agents to a subject to treat a disease. The two agents may be administered simultaneously, or sequentially in any order during the entire or portions of the treatment period. The at least two agents may be administered following the same or different dosing regimens.

As used herein, the term “glucocorticoid receptor” (“GR”) refers to the type II GR, a family of intracellular receptors which specifically bind to glucocorticoids such as cortisol and/or cortisol analogs such as dexamethasone (See, e.g., Turner & Muller, J. Mol. Endocrinol. Oct. 1, 2005 35 283-292). (The term “glucocorticoid” may be abbreviated as “GC”.) The type II glucocorticoid receptor is also referred to as the cortisol receptor. The term includes isoforms of GR, recombinant GR and mutated GR.

The term “glucocorticoid receptor modulator” (GRM) refers to any compound which modulates glucocorticoid binding to GR, or which modulates any biological response associated with the binding of GR to an agonist. For example, a GRM that acts as an agonist, such as dexamethasone, increases the activity of tyrosine aminotransferase (TAT) in HepG2 cells (a human liver hepatocellular carcinoma cell line; ECACC, UK). A GRM that acts as an antagonist, such as mifepristone, decreases the activity of tyrosine aminotransferase (TAT) in HepG2 cells. TAT activity can be measured as outlined in the literature by A. Ali et al., J. Med. Chem., 2004, 47, 2441-2452.

As used herein, the term “compound” is used to denote a molecular moiety of unique, identifiable chemical structure. A molecular moiety (“compound”) may exist in a free species form, in which it is not associated with other molecules. A compound may also exist as part of a larger aggregate, in which it is associated with other molecule(s), but nevertheless retains its chemical identity. A solvate, in which the molecular moiety of defined chemical structure (“compound”) is associated with a molecule(s) of a solvent, is an example of such an associated form. A hydrate is a solvate in which the associated solvent is water. The recitation of a “compound” refers to the molecular moiety itself (of the recited structure), regardless of whether it exists in a free form or an associated form.

As used herein, the term “composition” is intended to encompass a product comprising the specified ingredients such as the said compounds, their tautomeric forms, their derivatives, their analogues, their stereoisomers, their polymorphs, their deuterated species, their pharmaceutically acceptable salts, esters, ethers, metabolites, mixtures of isomers, their pharmaceutically acceptable solvates and pharmaceutically acceptable compositions in specified amounts, as well as any product which results, directly or indirectly, from combination of the specified ingredients in the specified amounts. Such term in relation to a pharmaceutical composition is intended to encompass a product comprising the active ingredient (s), and the inert ingredient (s) that make up the carrier, as well as any product which results, directly or indirectly, in combination, complexation or aggregation of any two or more of the ingredients, or from dissociation of one or more of the ingredients, or from other types of reactions or interactions of one or more of the ingredients. Accordingly, the pharmaceutical compositions of the present invention are meant to encompass any composition made by admixing compounds of the present invention with pharmaceutically acceptable carriers.

As used herein, the terms “pharmaceutically acceptable excipient” and “pharmaceutically acceptable carrier” refer to a substance that aids the administration of an active agent to—and absorption by—a subject and can be included in the compositions of the present invention without causing a significant adverse toxicological effect on the patient. These terms are intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. One of ordinary skill in the art will recognize that other pharmaceutical excipients are useful in the present invention.

### Pharmaceutical Compositions and Administration

In embodiments, the present methods and uses include administration of, and the use of, a pharmaceutical composition for treating CS, the pharmaceutical composition including a pharmaceutically acceptable excipient and a GRM such as, e.g., relacorilant. In some embodiments, the pharmaceutical composition includes a pharmaceutically acceptable excipient and a GRM. In preferred embodiments, the pharmaceutical composition includes relacorilant and a pharmaceutically acceptable excipient or excipients. Suitable formulations can be prepared and administered in a wide variety of oral, parenteral and topical dosage forms. For example, suitable pharmaceutical compositions containing relacorilant are disclosed in U.S. Pat. Nos. 11,464,764 and 11,925,626, hereby incorporated by reference in their entireties.

In embodiments, relacorilant is orally administered. In embodiments, relacorilant may be administered at a dose of, e.g., 10 milligrams (mg), 20 mg, 25 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg. In embodiments, relacorilant may be administered at daily doses of between about 10 milligrams (mg) to about 1000 mg, or between about 25 mg and about 800 mg. In embodiments, the daily dose of relacorilant is selected from 50 mg, 75 mg, 100 mg, 150 mg, 200 mg, 250 mg, 300 mg, 350 mg, 400 mg, 450 mg, and 500 mg. In embodiments, the daily dose of relacorilant is selected from 100 mg, 200 mg, 300 mg, and 400 mg.

### EXAMPLE

The following example is provided by way of illustration only and not by way of limitation. Those of skill in the art will readily recognize a variety of noncritical parameters which could be changed or modified to yield essentially similar results.

### Example. Relacorilant Effects on Tissue Lean Mass, Muscle Weakness, Body Weight, and Waist Circumference

In this study, patients suffering from Cushing's syndrome or Cushing's Disease (CS) were administered relacorilant for 22 weeks, then were eligible to be randomly assigned to receive further relacorilant, or placebo, for the following 12 weeks.

Criteria for patients to participate in this study included: age between 18-80 years old; suffering from Cushing's syndrome (CS); and suffering from hypertension, hyperglycemia (impaired glucose tolerance, or diabetes mellitus) or both.

152 patients were enrolled in the study and received 22 weeks of relacorilant administration (Open Label phase (OL)). Relacorilant administration began with a once-daily oral dose of 100 milligrams (mg) relacorilant, which dose was titrated upwardly in 100 mg dose increments up to a maximum of 400 mg once per day based on tolerability and efficacy. Patient blood pressure, blood glucose, and other clinical parameters were measured and used to determine efficacy of the relacorilant administration.

TABLE 1

| Patient Demographics & Baseline Characteristics | | | Open Label Phase | |
|---|---|---|---|---|
| | Hypertension | Hyperglycemia | Hypertension & | |
| Mean | only | only | hyperglycemia | Overall |
| (SD) | (n = 31) | (n = 50) | (n = 71) | (N = 152) |
| Age, yrs | 43.5 (11.6) | 54.1 (13.7) | 50.9 (12.6) | 50.4 (13.2) |
| Female, n (%) | 24 (77.4) | 42 (84.0) | 61 (85.9) | 127 (83.6) |
| Weight, kg | 95.2 (25.5) | 91.1 (21.4) | 95.0 (26.6) | 93.8 (24.7) |
| BMI, kg/m² | 33.4 (7.5) | 34.8 (7.9) | 35.3 (9.6) | 34.7 (8.6) |
| Waist circumference, cm | 112.8 (17.4) | 114.4 (14.7) | 116.1 (20.4) | 114.9 (18.0) |
| ACTH-dependent, n (%) | 23 (74.2) | 39 (78.0) | 56 (78.9) | 118 (77.6) |
| Plasma ACTH, pg/mL [n] | 67.7 (34.0) [23] | 74.9 (85.0) [39] | 78.1 (69.9) [56] | 74.9 (69.8) [118] |
| 24-h UFC, μg/d [n] | 191.2 (221.8) [18] | 148.0 (136.3) [26] | 257.9 (407.1) [39] | 209.0 (308.3) [83] |
| ACTH-independent, n (%) | 8 (25.8) | 11 (22.0) | 15 (21.1) | 34 (22.4) |
| Plasma ACTH, pg/mL [n] | 7.3 (4.8) [8] | 20.0 (26.6) [11] | 10.0 (6.2) [15] | 12.7 (16.2) [34] |
| 24-h UFC, μg/d [n] | 108.3 (88.9) [6] | 68.7 (67.9) [7] | 61.3 (30.5) [8] | 77.2 (64.0) [21] |
| Mean 24-h SBP (mm Hg) [n] | 138.1 (9.4) [30] | 124.6 (9.0) [47] | 141.6 (11.0) [71] | 135.5 (12.6) [148] |
| Mean 24-h DBP (mm Hg) [n] | 90.8 (5.7) [30] | 76.0 (7.3) [47] | 88.1 (7.6) [71] | 84.8 (9.4) [148] |
| HbA1c (%) | 5.4 (0.5) | 7.1 (1.6) | 7.2 (1.6) | 6.8 (1.6) |

^{a}Median ACTH was <5 pg/mL (hypertension only); 9 pg/mL (hyperglycemia only, hypertension and hyperglycemia, and overall).
ACTH, adrenocorticotropic hormone;
BMI, body mass index;
DBP, diastolic blood pressure;
mL, milliliter;
μg/d, micrograms per day;
OL, open-label phase;
pg, picogram;
SBP, systolic blood pressure;
SD, standard deviation;
UFC, urinary free cortisol.

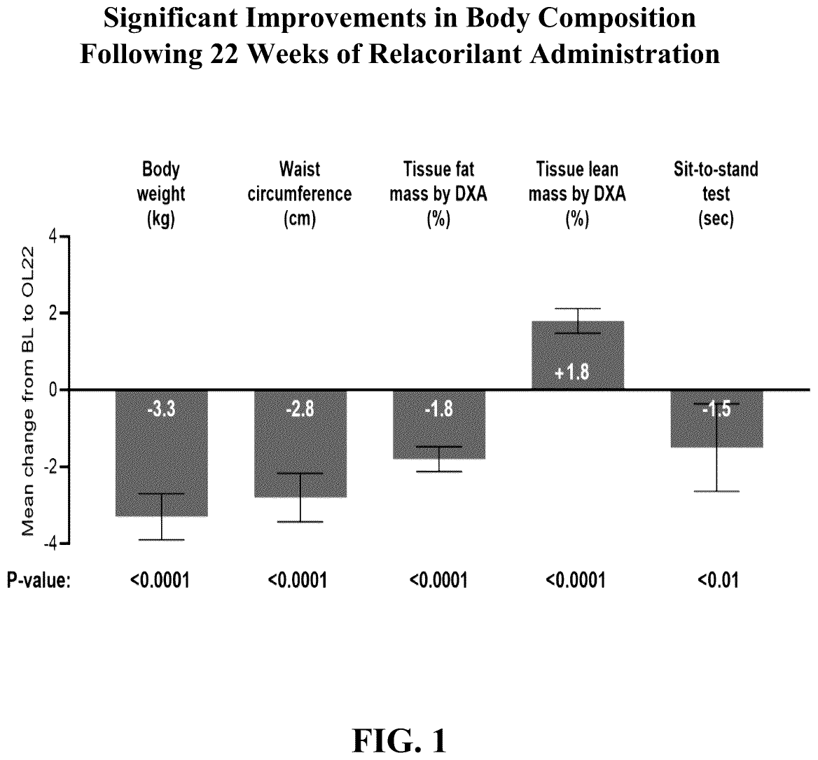

As shown in FIG. 1, which graphically depicts the loss of body weight experienced by CS patients who received 22 weeks of relacorilant administration (100 mg to 400 mg per day; the starting dose was 100 mg per day, administered for 2 weeks, followed by dose escalation of 100 mg per day every 4 weeks, up to a 400 mg/day maximum dose), patient mean body weight was reduced by 3.3 kilograms (kg) and waist circumference was reduced by 2.8 centimeters (cm). Tissue fat mass percentage (as measured by dual energy X-ray absorptiometry (DXA)) was reduced by 1.8%. In contrast, tissue lean mass percentage (as measured DXA) increased by 1.8% as compared to baseline following 22 weeks of relacorilant administration. All of the preceding measurements were significant with P values<0.0001 (Wilcoxon rank sum test P-values for the mean change from baseline).

In addition to the measured increase in tissue lean mass percentage, FIG. 1 shows that patients significantly reduced the time required to move from a sitting to a standing position (a 1.5 second (sec) reduction in the sit-to-stand test); this improvement was significant with a P value of 0.01 (Wilcoxon rank sum test P-values for the mean change from baseline). This reduced time in the sit-to-stand test is believed to indicate increased muscular strength, or a lessening or reversal of the muscular weakness associated with CS.

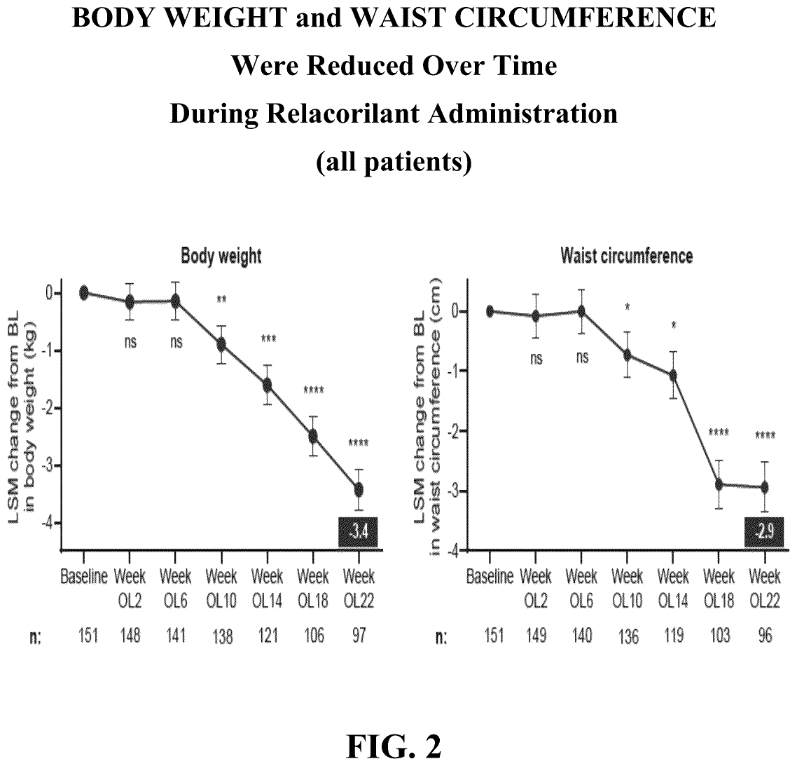

FIG. 2 shows the timecourse of the reduction in body weight (left-hand graph) and in waist circumference (right-hand graph) over the 22 weeks of relacorilant administration. By week 10 of relacorilant administration, CS patients had lost significant amounts of weight, and significantly reduced their waist circumference, as compared to baseline weight and baseline waist circumference measurements.

These results show that relacorilant administration is effective to improve tissue lean mass percentage and improve muscle strength (as indicated by reduced time required to stand up from a sitting position) in patients suffering from CS; and to reduce fat, body weight, and waist circumference in patients suffering from CS. “Lean mass” in the following table indicates tissue lean mass. These results are tabulated in Table 2.

TABLE 2

| | | Visit | Change from | P- |
|---|---|---|---|---|
| Mean (SD) | Baseline | OL22 | Baseline to OL22 | value |
| Fat mass by | 46.4 ± 8.4 | 43.9 ± 7.6 | −1.8 ± 2.7 | <0.0001 |
| DXA, % | n = 131 | n = 76 | n = 71 | |
| Lean mass by | 53.6 ± 8.4 | 56.1 ± 7.6 | +1.8 ± 2.7 | <0.0001 |
| DXA, % | n = 131 | n = 76 | n = 71 | |
| Sit-to-stand test, | 16.4 ± 11.5 | 14.1 ± 9.0 | −1.5 ± 10.5 | 0.0032 |
| total time, sec | n = 141 | n = 89 | n = 85 | |

DXA, dual energy X-ray absorptiometry

Patients who met the following criteria after 22 weeks of relacorilant administration were eligible for the “Random Withdrawal” extension study:
Hypertension control (for patients with hypertension) is defined as: ≥5 millimeter (mm) mercury (Hg) decrease in mean systolic blood pressure (SBP) and/or diastolic blood pressure (DBP), without worsening of either, based on 24 hour (24-h) ambulatory blood pressure measurement (ABPM);
Hyperglycemia control (for patients with impaired glucose tolerance) is defined as: two hour (2-h) oral glucose tolerance test (oGTT) glucose normalization (where glucose normalization is defined as oGTT of less than 140 mg/dL glucose).
Hyperglycemia control (for patients with diabetes) is defined as: at least one of a) HbA1c decrease by ≥0.5%; b) 2-h oGTT glucose normalization (<140 mg/dL) or decrease by ≥50 mg/dL; c) total daily insulin dose decrease by ≥25% and HbA1c unchanged or decreased.

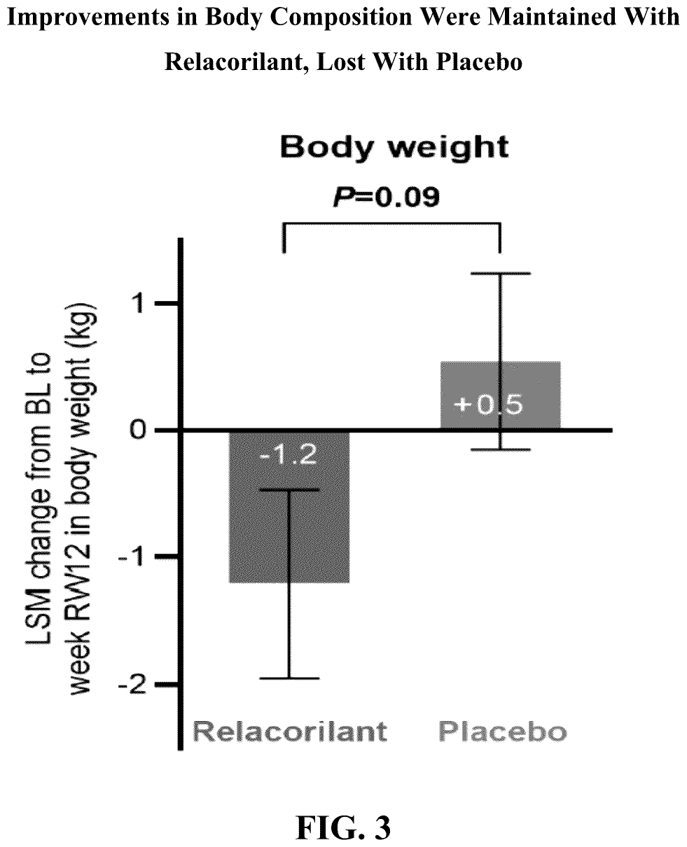

Improvements in body composition, such as those shown in FIG. 1 and FIG. 2, were maintained in patients who continued to receive relacorilant administration during the 12 week Randomized Withdrawal phase following the 22 week Open Label phase, but were not maintained in patients who received placebo for the 12 weeks following the 22 weeks of relacorilant administration during the Open Label phase. As shown in FIG. 3, patients who continued to receive relacorilant maintained a 1.2% reduction in body weight (as compared to baseline weight measured prior to beginning relacorilant administration), while patients who received placebo (and no longer received relacorilant) during the 12 week Random Withdrawal phase showed a 0.5% increase in body weight (as compared to baseline weight measured prior to beginning relacorilant administration).

Out of 152 patients enrolled in the Open Label (OL) Phase, 95 (65%) completed the OL phase. In the OL phase, 66.7% (n=44) achieved hypertension control, and 57.5% (n=42) achieved hyperglycemic control. Of the 95 patients who completed the OL phase, 62 (65.3%) entered the Randomized Withdrawal (RW) phase. 58 (93.5%) of these patients finished the RW phase. Characteristics of patients who enrolled in the Randomized Withdrawal phase are presented in Table 3.

TABLE 3
Randomized Withdrawal Phase

| | Relacorilant | | Placebo | |
|---|---|---|---|---|
| Mean (SD) | (n = 30) | | (n = 32) | |
| Age, yrs | 46.6 | (11.0) | 48.8 | (14.4) |
| Female, n (%) | 22 | (73.3) | 26 | (81.3) |
| Weight, kg | 93.3 | (27.4) | 88.6 | (21.1) |
| BMI, kg/m² | 33.3 | (7.6) | 32.6 | (6.5) |
| Waist circumference, cm | 113.8 | (17.7) | 108.9 | (17.1) |
| ACTH-dependent, n (%) | 26 | (86.7) | 23 | (71.9) |
| Plasma ACTH, pg/mL | 91.7 | (85.7) | 71.7 | (74.7) |
| 24-h UFC, μg/d | 257.1 | (449.1) | 301.3 | (287.9) |
| ACTH-independent, n (%) | 4 | (13.3) | 9 | (28.1) |
| Plasma ACTH, pg/mL | 5.9 | (2.3) | 10.0 | (9.0) |
| 24-h UFC, μg/d | 66.9 | (36.8) | 142.2 | (194.1) |

ACTH, adrenocorticotropic hormone; BMI, body mass index; OL, open-label phase; RW, randomized withdrawal; SD, standard deviation; UFC, urinary free cortisol.

The improvements in body composition were maintained in the patients who continued to receive relacorilant administration during the 12 weeks of the Randomized Withdrawal phase, but these improvements were not maintained in the patients who did not receive relacorilant, but instead received placebo administration during the 12 weeks of the Randomized Withdrawal phase.

FIG. 3 shows that the improvements in body weight in patients who continued to receive relacorilant administration during the 12 week Randomized Withdrawal phase following the 22 week Open Label phase were maintained. These improvements in body weight were not maintained in patients who received placebo for the 12 weeks following the 22 weeks of relacorilant administration during the Open Label phase. These results show that relacorilant administration is effective to reduce body weight in patients suffering from CS.

These results regarding continued improvements in body composition with continued relacorilant administration are provided in TABLE 4. Similar trends observed across measures of body composition. Those who switched to placebo experienced a deterioration in body composition. In contrast, trends toward further improvement were observed in the relacorilant arm.

TABLE 4
Improvements in Body Composition
Were Maintained With Relacorilant

| | |
|---|---|
| Relacorilant | Placebo |
| (n = 30) | (n = 32) |

Change from RW baseline to week RW12 in all patients in the RW phase:

| | | |
|---|---|---|
| Waist circumference | | |
| n | 26 | 30 |
| Mean (SD), cm | −1.2 (3.7) | +3.8 (10.4) |
| Wilcoxon signed rank sum P-value* | ns | 0.008 |
| Tissue fat mass | | |
| n | 17 | 22 |
| Mean (SD), % | −0.2 (1.7) | +1.6 (1.8) |
| Wilcoxon signed rank sum P-value* | ns | 0.0002 |
| Tissue lean mass | | |
| n | 17 | 22 |
| Mean (SD), % | +0.2 (1.7) | −1.6 (1.8) |
| Wilcoxon signed rank sum P-value* | ns | 0.0002 |

*Wilcoxon signed-rank test P-values within each treatment arm
BL, baseline; LSM, least squares mean; ns, not significant (P ≥ 0.05); RW, randomized withdrawal; SE, standard error.

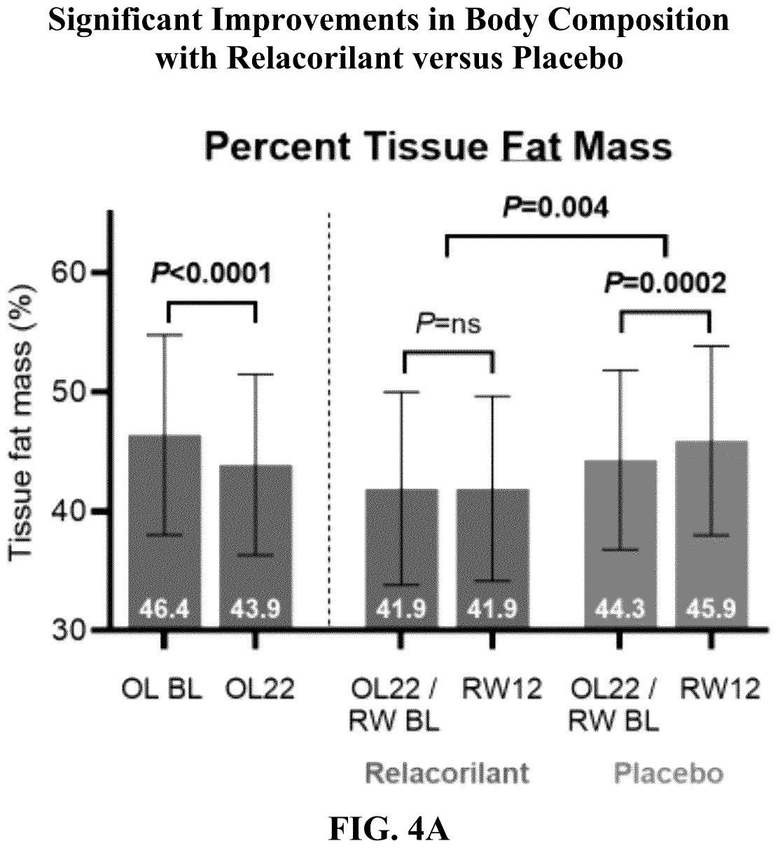

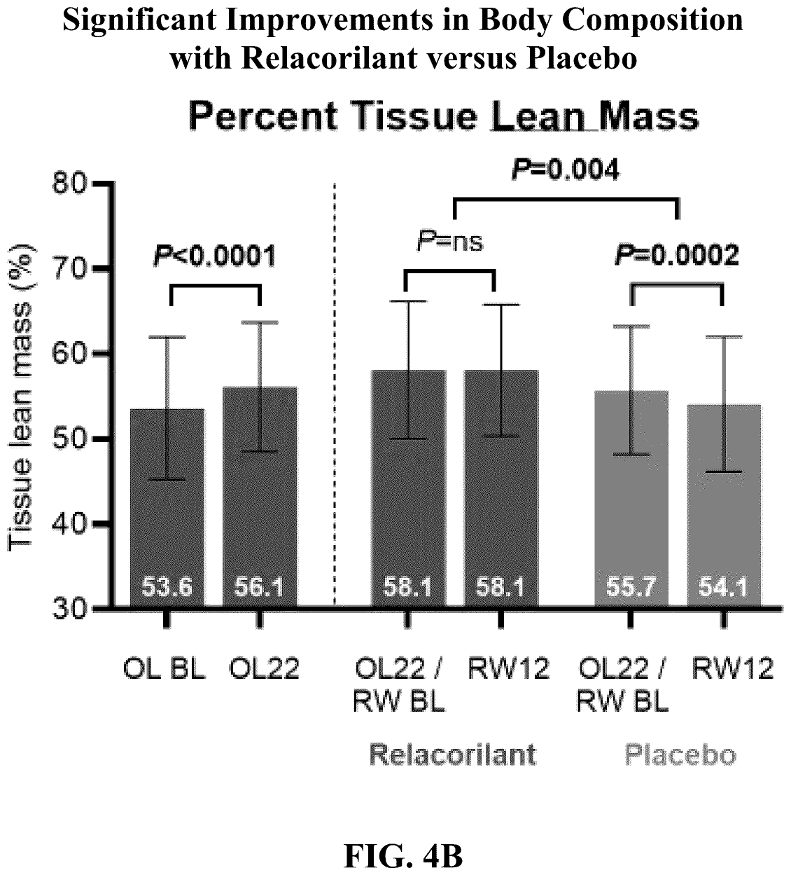

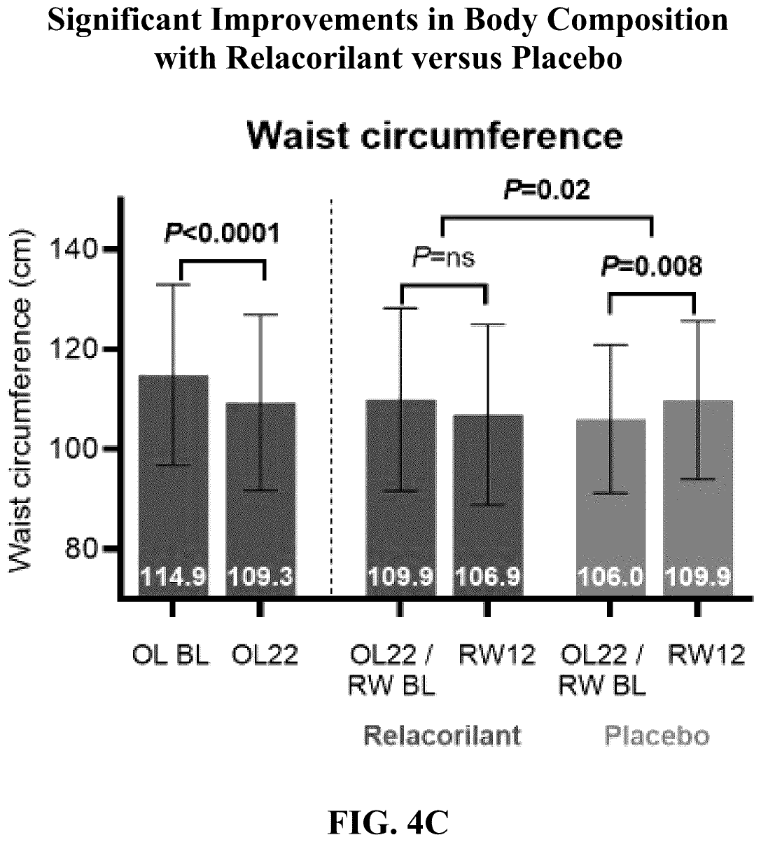

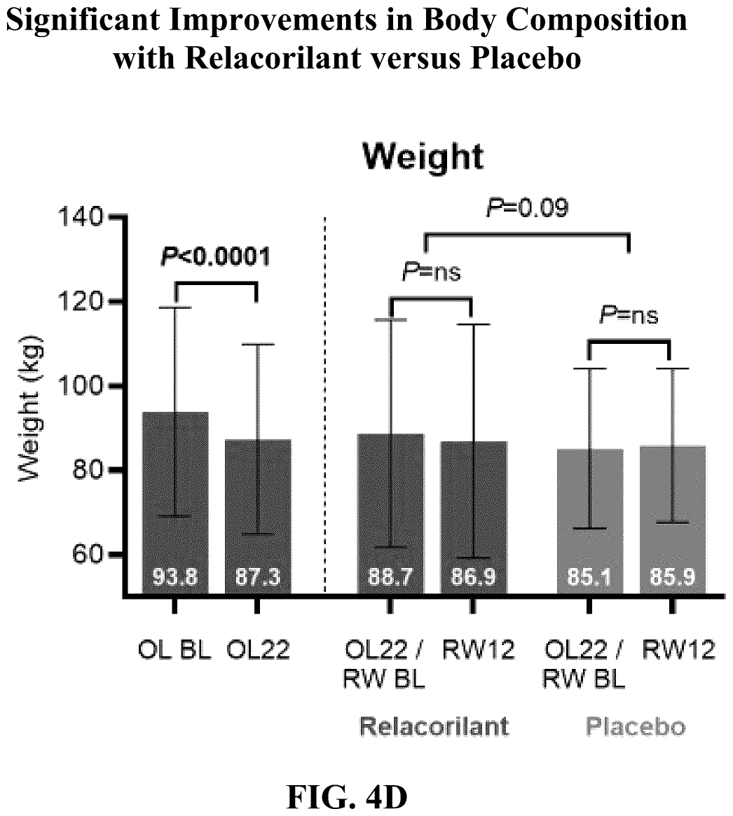

FIGS. 4A, 4B, 4C, and 4D illustrate the significant reduction in percent tissue fat mass with a corresponding increase in percent tissue lean mass with 22 weeks of daily relacorilant administration (during the Open Label (OL) phase of the study). Similarly, patient waist circumference and patient body weight were significantly reduced during the OL phase. (In these figures, BL indicates baseline; DXA indicates dual X-ray absorptiometry; OL indicates Open Label phase of the study; RW indicates Randomized Withdrawal phase of the study; Error bars indicate standard deviation; tissue fat mass and tissue lean mass were measured by DXA; Wilcoxon rank sum test P-values for the mean change from baseline are shown.)

A Randomized Withdrawal (RW) phase followed the 22 weeks of daily relacorilant administration in the OL phase, in which patients were randomly assigned to continue daily relacorilant administration or to daily placebo administration. The reduction in percent tissue fat mass and the increase in percent lean mass were maintained in patients receiving relacorilant during the RW phase; in contrast, significant deterioration in percent tissue fat mass (increase) and in percent tissue lean mass (decrease) was observed in patients who received placebo during the RW phase. No significant change in patient waist circumference or in patient body weight was observed in patients receiving relacorilant during the RW phase.

As shown in FIG. 4A, tissue fat mass percent was significantly reduced at week 22 of the Open Label (OL) phase as compared to baseline (BL) (P<0.0001). As measured at the end of the 12 week Randomized Withdrawal (RW) phase, the reduction in tissue fat mass percent was maintained in patients who continued to receive relacorilant, but was not maintained in patients who received placebo in the RW phase that followed the 22 week OL phase.

As shown in FIG. 4B, tissue lean mass percent was significantly increased at week 22 of the OL phase as compared to BL (P<0.0001). As measured at the end of the 12 week RW phase, the increase in tissue lean mass percent was maintained in patients who continued to receive relacorilant, but was not maintained in patients who received placebo in the RW phase that followed the 22 week OL phase.

As shown in FIG. 4C, patient waist circumference was significantly reduced at week 22 of the OL phase as compared to BL (P<0.0001). As measured at the end of the 12 week RW phase, the reduction in waist circumference was maintained in patients who continued to receive relacorilant administration, but was not maintained in patients who received placebo in the RW phase that followed the 22 week OL phase.

As shown in FIG. 4D shows patient body weight was significantly reduced at week 22 of the OL phase as compared to BL (P<0.0001). As measured at the end of the 12 week RW phase, the reduction in body weight was maintained in patients who continued to receive relacorilant administration during the 12 week RW that followed the 22 week OL phase. The body weight reduction was also maintained in patients who received placebo during the 12 week RW phase.

All patents, patent publications, publications, and patent applications cited in this specification are hereby incorporated by reference herein in their entireties as if each individual publication or patent application were specifically and individually indicated to be incorporated by reference. In addition, although the foregoing invention has been described in some detail by way of illustration and example for purposes of clarity of understanding, it were readily apparent to those of ordinary skill in the art in light of the teachings of this invention that certain changes and modifications may be made thereto without departing from the spirit or scope of the appended claims.

1. A method of treating a patient suffering from endogenous hypercortisolism (CS) effective to: a) reduce loss in tissue lean mass over time of the patient as compared to the average loss of tissue lean mass over time of CS patients who have not received treatment according to the present method of treating CS or as compared to the baseline loss of tissue lean mass over time of said CS patient determined prior to beginning the present method of treating CS, or
b) increase the tissue lean mass of the patient as compared to baseline tissue lean mass of the patient prior to beginning the present method of treating CS, or
c) increase muscular strength in the patient as compared to the baseline level of muscular strength, wherein said baseline level of muscular strength is determined prior to beginning the present method of treating CS, or
d) to reduce body weight as compared to the baseline level of body weight in the patient, wherein said baseline level of body weight is determined prior to beginning the present method of treating CS,
e), to reduce waist circumference as compared to the patient's baseline waist circumference determined prior to beginning the present method of treating CS,
or f) combinations thereof,
the method comprising:
Administering an effective amount of the heteroaryl ketone fused azadecalin compound relacorilant, ((R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridine-2-yl)methanone), which has the structure: 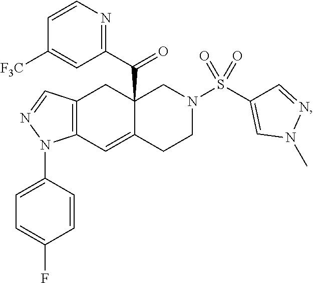 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]
effective to a) reduce the loss of tissue lean mass of said patient as compared to the average loss of tissue lean mass of a CS patient who has not received relacorilant administration, or b) increase the tissue lean mass of the patient as compared to baseline levels of tissue lean mass, or c) increase muscular strength in the patient as compared to baseline levels of muscular strength, or d) reduce body weight as compared to baseline levels of body weight in the patient, or e) to reduce the patient's waist circumference as compared to the patient's baseline waist circumference, or f) combinations thereof.

2. The method of claim 1, wherein the effective amount of relacorilant is an amount selected from the group consisting of a) between about 50 milligrams per day (mg/day) and about 800 mg/day, b) between about 50 mg/day and about 500 mg/day, and c) between about 400 mg/day and about 800 mg/day.

3. The method of claim 1, wherein said administration is oral administration.

4. The method of claim 3, wherein said oral administration is with food.

5. The method of claim 3, wherein said oral administration is without food.

6. The method of claim 1, wherein said relacorilant administration comprises 22 weeks of daily relacorilant administration.

7. The method of claim 1, wherein said time period over which loss in tissue mass is determined is a period of time selected from one month, two months, three months, six months, nine months, and one year.

8. The method of claim 3, wherein said time period over which loss in tissue mass is determined is a period of time selected from one month, two months, three months, six months, nine months, and one year.

9. The method of claim 1, wherein said tissue lean mass of the patient is increased by about 1.8% as compared to baseline tissue lean mass of the patient.

10. The method of claim 3, wherein said tissue lean mass of the patient is increased by about 1.8% as compared to baseline tissue lean mass of the patient.

11. The method of claim 1, wherein said muscular strength is measured by a reduction in the time required by the patient to stand up from a sitting position, wherein said time required by the patient to stand up from a sitting position following relacorilant administration is reduced by about 1.5 seconds as compared to the time required prior to relacorilant administration.

12. The method of claim 3, wherein said muscular strength is measured by a reduction in the time required by the patient to stand up from a sitting position, wherein said time required by the patient to stand up from a sitting position following relacorilant administration is reduced by about 1.5 seconds as compared to the time required prior to relacorilant administration.

13. The method of claim 1, wherein said body weight of said patient is reduced by at least about 2% as compared to the baseline body weight of the patient.

14. The method of claim 3, wherein said body weight of said patient is reduced by at least about 2% as compared to the baseline body weight of the patient.

15. The method of claim 1, wherein said body weight of said patient is reduced by at least about 3.4% as compared to the baseline body weight of the patient.

16. The method of claim 3, wherein said body weight of said patient is reduced by at least about 3.4% as compared to the baseline body weight of the patient.

17. The method of claim 1, wherein said waist circumference is reduced by at least about 1% as compared to the baseline waist circumference of the patient.

18. The method of claim 3, wherein said waist circumference is reduced by at least about 1% as compared to the baseline waist circumference of the patient.

19. The method of claim 1, wherein said waist circumference is reduced by about 2.9% as compared to the baseline waist circumference of the patient.

20. The method of claim 3, wherein said waist circumference is reduced by about 2.9% as compared to the baseline waist circumference of the patient.

21. The method of claim 1, wherein tissue lean mass in the patient is not reduced.

22. The method of claim 3, wherein tissue lean mass in the patient is not reduced.

23. The method of claim 1, wherein tissue lean mass in the patient is increased.

24. The method of claim 3, wherein tissue lean mass in the patient is increased.
